(19) United States
(12) Patent Application Publication
(10) Pub. No.: US 2011/0137266 A1
(43) Pub. Date: Jun. 9, 2011

(54) Catheter

(76) Inventor: Michael Schlitt, Burien, WA (US)

(21) Appl. No.: 12/632,630

(22) Filed: Dec. 7, 2009

(51) Int. Cl. A61M 25/06

(52) U.S. Cl. 604/272

(57) ABSTRACT

A catheter fabricated of incineratable polymeric material, including a solid pusher rod closely mated to the interior of the cannula, engagable with the front tapered end thereof to strengthen the cannula during insertion into the body.

### TECHNICAL FIELD

This invention relates to catheters, and more particularly to a catheter fabricated out of a polymeric material capable of being incinerated, wherein the outer cannula is sharpened to facilitate penetration of the skin and blood vessel, and includes exterior bores leading to a hollow interior, wherein it is inserted into the vein and left behind having been placed by means of an inner rod, which replaces the usual cannula, which is a stiffening and pushing device which is then removed.

### BACKGROUND OF THE INVENTION

Metal hypodermic needles and catheters have been available and have not changed in basic design in a number of years. These instruments have many disadvantages in that they are dangerous, they can be involved in the transmission of deadly diseases, such as AIDS, and the metal needles and lancets are extremely difficult to destroy thereby posing an ongoing disposal and health problem.

There have been many attempts to solve the problems inherent with the metal needles. Such attempts have taken two separate routes; the first is the utilization of a plastic for the metal in the needle material and the second is a guard to shield the needle. These attempts to solve the needle problem have not been successful. The plastic needles are thin and, if hard enough to puncture the skin, are too brittle and shatter. More flexible plastic needles lack the requisite stiffness to penetrate the skin and/or vein. Needle guards themselves have proven to be unwieldy or too costly to use or ineffective in preventing accidental punctures.

The art known to the inventor includes the following:

U.S. Pat. No. 2,512,568 granted to Saffir on Jun. 20, 1950, which discloses the concept of a non-metallic hypodermic injection device or cannula;

U.S. Pat. No. 2,954,768 granted to Hamilton on Oct. 4, 1960, which discloses a puncture point fabricated of a plastic material;

U.S. Pat. No. 4,767,407 granted to Foran on Aug. 30, 1988, which discloses a hypodermic needle primarily designed for positioning a catheter when the tip structure is especially suitable for use with a patient who has venous constriction or collapse;

U.S. Pat. No. 4,838,877 granted to Massau on Jun. 13, 1989 discloses a polymeric hypodermic device including a novel injection end;

U.S. Pat. No. 5,275,616 granted to Fowler on Jan. 4, 1994, discloses a plunger-type mechanism for insertion of a vessel plug into the body of a patient;

U.S. Pat. No. 5,607,401 granted to Humphrey on Mar. 4, 1997, which discloses augmented polymeric hypodermic needles and lancets wherein the devices are stiffened by augmenting means so that they are able to pierce the skin;

U.S. Pat. No. 6,564,078 granted to Marino et al. on May 13, 2003, which discloses an expandable tip cannula system including an expandable tip comprising a plurality of generally triangular-shaped petals, wherein each petal comprises a nerve sensing electrode;

U.S. patent publication No. US2003/0028154 to Ross on Feb. 6, 2003, which discloses a polymeric hypodermic needle and the process for making an all plastic one-piece hypodermic;

### SUMMARY OF THE INVENTION

With the above-noted prior art in mind, it is the goal of the present invention to provide a cannula that is fabricated of a combustible material and is thin enough to penetrate the skin/vein without causing undue trauma during insertion, said needle being stiffened by a pushrod which replaces the usual cannula used to insert the outer cannula and then be removed during the usage thereof.

It is the further goal of the present invention to provide a catheter that is capable of being incinerated and which avoids the problems and risks associated with a metallic needle or catheter.

### BRIEF DESCRIPTION OF THE DRAWINGS

FIG. 1 is an isometric view of the inventive catheter with the pushrod removed.

FIG. 2 is an isometric view of the inventive catheter with the pushrod in place for insertion of the catheter.

FIG. 3 is a perspective view the inventive catheter with the pushrod being removed.

FIG. 4 is a sectional view of along lines 4-4 of FIG. 2.

FIG. 5 is a pictorial view of the catheter in use.

### BEST MODE FOR CARRYING OUT THE INVENTION

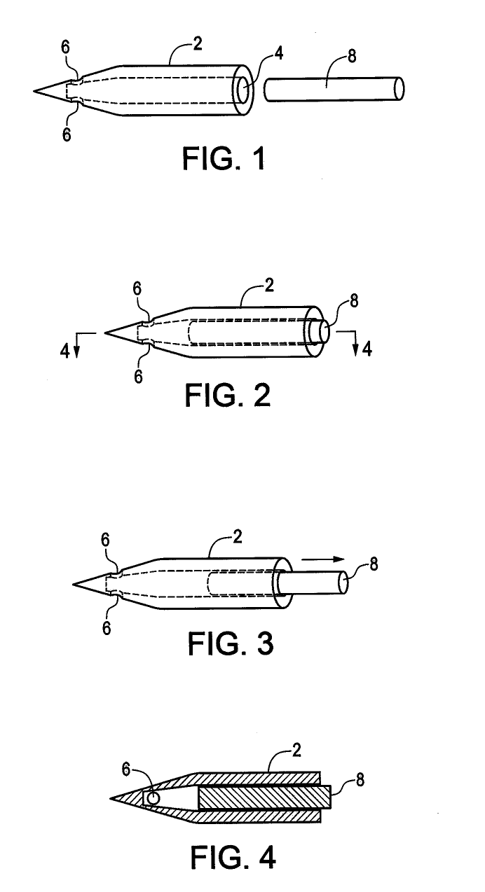

As seen in FIG. 1, the catheter comprises an outer cannula 2, including a hollow interior 4 and openings 6 adjacent the pointed skin penetrating end which extend from the exterior of the cannula 2 to the interior 4. Because it is proposed that the outer cannula be fabricated of a preferably transparent polymeric material, and being of a dimension to minimize trauma upon insertion, the inner cannula or pushrod 8 comprises a stiffening rod which closely mates with the interior bore 4 in the outer cannula 2, such that the outer cannula can be inserted through the skin and flesh and into the vein of the subject.

As seen in FIGS. 2 and 4, wherein the outer cannula 2 is being inserted, the pushrod 8 is adjacent and abuts the forwardmost end of the bore 4, engaging the tapered inner wall, while strengthening and making the outer cannula 2 rigid enough to penetrate the necessary material.

As seen in FIG. 3, the outer cannula 2 is in position in the vein and the inner cannula 8 or pushrod is being removed.

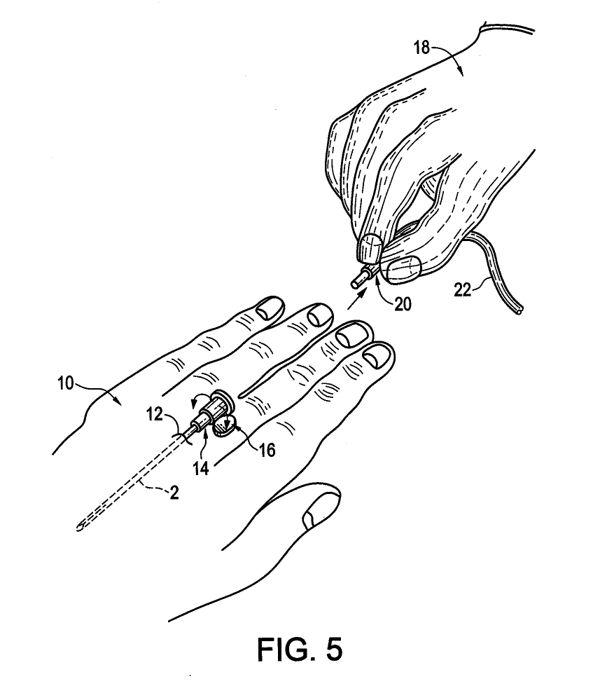

Now viewing FIG. 5 wherein there is shown a hand and wherein the cannula 2 has been inserted through the skin at 12 and into a vein for providing or removing fluid from the vein. The cannula 2 includes an outer flange 14 of greater radius than the shaft of the cannula and may in fact include a tab 16 for aid in securing the cannula to the patient's hand. Also seen in this view is a second hand 18 about to connect a hollow tube 20 into the cannula to either supply or remove fluid which is transported via tube 22.

As can be seen, the present invention resolves the problem of metallic instruments which are dangerous and difficult to destroy by providing a cannula fabricated of polymeric material. The cannula, which is stiffened by a rod insertion, performs as well as the prior art metallic devices, and yet can be easily destroyed by incineration, thereby removing the danger of spreading contamination and disease.

Although a preferred embodiment has been disclosed for purposes of illustration, it should be understood that various changes and modifications and substitutions could be made in the preferred embodiment without departing from the spirit of the invention as defined by the claims which follow:

What is claimed is:

1. A hollow catheter for delivering or receiving fluid from a vein, comprising:
a hollow, cylindrical, elongated, thin, incineratable main body portion including an interior wall having a first end for piercing, penetrating and temporarily indwelling in a vein, said first end including a solid piercing point and a plurality of openings extending from adjacent the point to the hollow interior and a second end adapted to mate with a tube for delivery or receipt of the fluid; and
a cylindrical, rigid pusher rod closely mated to the interior wall of said body portion, said pusher rod inserted within said main body portion during the placement thereof within the vein and removed prior to the attachment of a tube.

2. (canceled)

3. A hollow catheter fabricated of material capable of being incinerated, comprising:
an elongated, hollow main body portion with an inner wall, including a pointed solid, uniformly tapered first end including openings extending from the tapered surface to the hollow interior and a second end adapted to receive the end of a mating tube; and
an insertable a cylindrical pusher rod closely mated to the inner wall of the catheter and used for inserting the first end of the catheter into a receiving vein and then removed.
